(19) United States
(12) Patent Application Publication
(10) Pub. No.: US 2026/0199001 A1
(43) Pub. Date: Jul. 16, 2026

(54) SYSTEMS AND METHODS FOR ELECTROPORATION USING WAVEFORMS THAT REDUCE ELECTRICAL STIMULATION

(71) Applicant: St. Jude Medical, Cardiology Division, Inc., St. Paul, MN (US)

(72) Inventors: Leonard Jan van Schelven, Houten (NL); Albert Harald Westra, Utrecht (NL)

(21) Appl. No.: 19/314,431

(22) Filed: Aug. 29, 2025

Related U.S. Application Data
(63) Continuation of application No. 18/159,329, filed on Jan. 25, 2023.
(60) Provisional application No. 63/304,321, filed on Jan. 28, 2022.

(51) Int. Cl. A61B 18/14; A61B 18/00

(52) U.S. Cl. CPC A61B 18/1492; A61B 2018/00613; A61B 2018/00732; A61B 2018/00767

(57) ABSTRACT

A pulse generator for use with an electroporation system is provided. The pulse generator is configured to be coupled to a catheter including a plurality of electrodes and is configured to generate a waveform to be delivered using at least one of the plurality of electrodes. The waveform includes a pulse train having positive and negative pulses, wherein an average charge over the pulse train is zero.

### CROSS-REFERENCE TO RELATED APPLICATIONS

This application is a continuation of U.S. Patent Application No. 18/159,329 filed January 25, 2023, which claims the benefit of priority to U.S. Provisional Patent Application No. 63/304,321, filed January 28, 2022, the entire contents and disclosure of which are hereby incorporated by reference herein.

### FIELD OF THE DISCLOSURE

The present disclosure relates generally to waveform generation. In particular, the present disclosure relates to electroporation waveforms that reduce electrical stimulation.

### BACKGROUND

It is generally known that ablation therapy may be used to treat various conditions afflicting the human anatomy. For example, ablation therapy may be used in the treatment of atrial arrhythmias. When tissue is ablated or at least subjected to ablative energy generated by an ablation generator and delivered by an ablation applicator (e.g., a catheter), lesions form in the tissue. The local destruction of cardiac tissue can correct conditions such as atrial arrhythmia (including, but not limited to, ectopic atrial tachycardia, atrial fibrillation, and atrial flutter.

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a non-thermal ablation technique that involves applying strong electric fields that induce pore formation in the cellular membrane. The electric field may be induced by applying a relatively short duration pulse which may last, for instance, from a nanosecond to several milliseconds. Such a pulse may be repeated to form a pulse train. When such an electric field is applied to tissue in an in vivo setting, the cells in the tissue are subjected to a trans-membrane potential which creates pores opening the cell wall. Electroporation may be reversible (i.e., the temporally opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction by causing irreversible electroporation.

In electroporation applications, different waveforms may be used to achieve different goals. For example, some waveforms may result in larger or smaller lesion sizes than other waveforms. Further, some waveforms result in higher or lower overall energy delivery than other waveforms (less overall energy delivery generally corresponds to less heating of the target tissue). As another example, some waveforms may be more likely to induce muscular contractions in a patient. Generally, however, it is desirable to reduce electrical stimulation resulting from waveforms, in order to have little to no skeletal muscle recruitment (i.e., avoiding muscle contractions.

### BRIEF SUMMARY OF THE DISCLOSURE

In one aspect, a pulse generator for use with an electroporation system is provided. The pulse generator is configured to be coupled to a catheter including a plurality of electrodes and is configured to generate a waveform to be delivered using at least one of the plurality of electrodes. The waveform includes a pulse train having positive and negative pulses, wherein an average charge over the pulse train is zero.

In another aspect, a pulse generator for use with an electroporation system is provided. The pulse generator is configured to be coupled to a catheter including a plurality of electrodes and is configured to generate a waveform to be delivered using at least one of the plurality of electrodes. The waveform includes a pulse train having positive and negative pulses, wherein the pulse train includes a first pulse, a last pulse, and at least one intermediate pulse between the first pulse and the last pulse, and wherein, to facilitate reducing a maximum absolute charge of the pulse train, the first and last pulses have at least one of a different amplitude and a different pulse width than the at least one intermediate pulse.

In yet another aspect, a method for controlling an electroporation system is provided. The method includes generating, using a pulse generator, a waveform including a pulse train having positive and negative pulses, wherein an average charge over the pulse train is zero, and delivering, using one or more electrodes on a catheter coupled to the pulse generator, the generated waveform to target tissue.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy.

FIGS. 2A-2C are embodiments of different waveforms that may be delivered using the system shown in FIG. 1.

FIGS. 3A and 3B are embodiments of different waveforms that may be delivered using the system shown in FIG. 1.

FIG. 4 is an embodiment of a waveform that may be delivered using the system shown in FIG. 1.

### DETAILED DESCRIPTION OF THE DISCLOSURE

The present disclosure provides a pulse generator for use with an electroporation system is provided. The pulse generator is configured to be coupled to a catheter including a plurality of electrodes and is configured to generate a waveform to be delivered using at least one of the plurality of electrodes. The waveform includes a pulse train having positive and negative pulses with at least one of a reduced maximum absolute charge and a zero average charge over the pulse train.

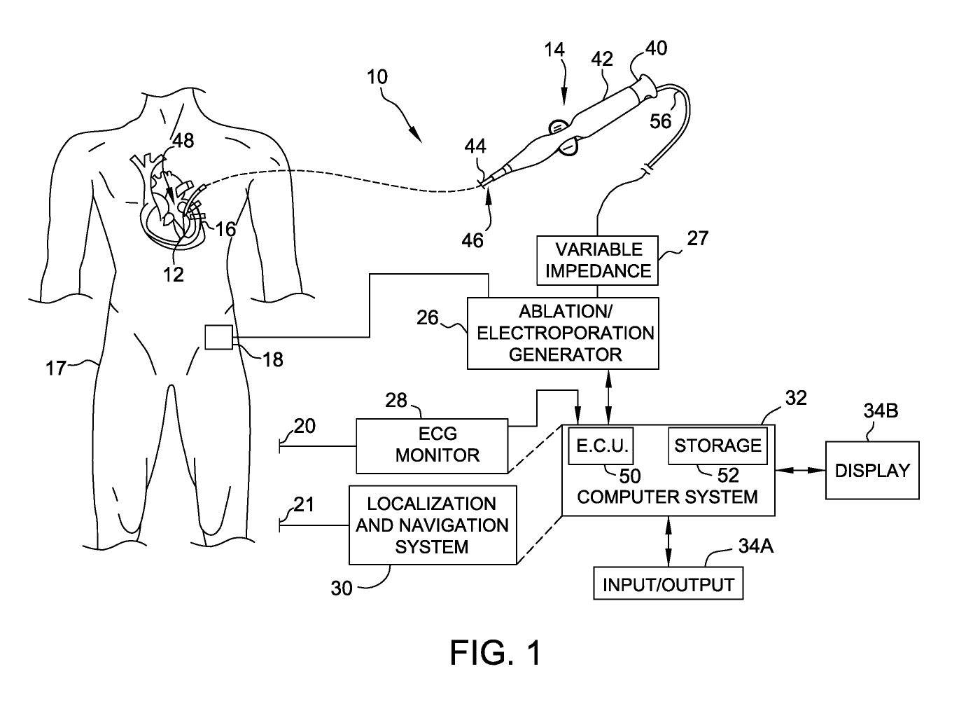

FIG. 1 is a schematic and block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

System 10 may be used for irreversible electroporation (IRE) to destroy tissue. In particular, system 10 may be used for electroporation-induced therapy that includes delivering electrical current in such a manner as to directly cause an irreversible loss of plasma membrane (cell wall) integrity leading to its breakdown and cell destruction. This mechanism of cell destruction may be viewed as an “outside-in” process, meaning that the disruption of the outside wall of the cell causes detrimental effects to the inside of the cell. Typically, for classical plasma membrane electroporation, electric current is delivered as a pulsed electric field in the form of short-duration pulses (e.g., having a 500 nanosecond (ns) to 20 microsecond (µs) duration) between closely spaced electrodes capable of delivering an electric field strength of about 0.1 to 1.0 kilovolts/centimeter (kV/cm). System 10 may be used for high output (e.g., high voltage and/or high current) electroporation procedures. Further, system 10 may be used with a loop catheter and/or with a basket catheter.

In one embodiment, stimulation is delivered selectively (e.g., between pairs of electrodes in a bipolar mode) on catheter 14. Further, the electrodes on catheter 14 may be switchable between being connected to a 3D mapping system and being connected to an electroporation generator.

It should be understood that while the energization strategies are described as involving DC electroporation pulses (i.e., square wave pulses), embodiments may use variations and remain within the spirit and scope of the disclosure. For example, AC pulses (i.e., sinusoidal pulses), RF pulses, sequences of RF bursts, asymmetric pulses, and combinations may be used. In addition, the embodiments described herein may be implemented in medical therapies other than electroporation therapy (e.g., electro-surgery, electrochemical therapy, cancer therapy, diathermy, etc.

For example, although the methods and systems disclosed are described herein in terms of an electroporation application, those of skill in the art will appreciate that the techniques described herein may be implemented in any suitable application.

Further, it should be understood that the mechanism of cell destruction in electroporation is not primarily due to heating effects, but rather to cell membrane disruption through application of a high-voltage electric field. Thus, electroporation may avoid some possible thermal effects that may occur when using other energy applications. This “cold therapy” thus has desirable characteristics.

With this background, and now referring again to FIG. 1, system 10 includes a catheter electrode assembly 12 including at least one catheter electrode. Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues (e.g., renal tissue, tumors, etc.

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation therapy, generator 26 may be configured to produce an electric current that is delivered via electrode assembly 12 as a pulsed electric field in the form of short-duration square wave pulses (e.g., of nanoseconds to several milliseconds duration, or any duration suitable for electroporation) between closely spaced electrodes capable of delivering an electric field strength (i.e., at the tissue site) of about 0.1 to 1.0 kV/cm. The amplitude and pulse width needed for irreversible electroporation are inversely related. That is, as pulse widths are decreased, the amplitude may generally be increased to achieve pore formation.

Electroporation generator 26, sometimes also referred to herein as a DC energy source, is configured to generate a series of DC energy pulses (i.e., square wave pulses) that produce current in two directions (i.e., positive and negative pulses). In other embodiments, electroporation generator 26 is any suitable type of electroporation generator. In some embodiments, electroporation generator 26 is configured to output energy in DC pulses at selectable energy levels, such as fifty joules, one hundred joules, two hundred joules, and the like. Other embodiments may have more, or fewer, energy settings and the values of the available setting may be the same or different. For successful electroporation, some embodiments utilize a two hundred joule output level. For example, electroporation generator 26 may output a DC pulse having a peak magnitude from about 300 Volts (V) to about 3,200 V at the two hundred joule output level. Other embodiments may output any other suitable positive or negative voltage.

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

With continued reference to FIG. 1, as noted above, catheter 14 may include functionality for electroporation and in certain embodiments also additional ablation functions (e.g., RF ablation). It should be understood, however, that in those embodiments, variations are possible as to the type of ablation energy provided (e.g., cryoablation, ultrasound, etc.

In the illustrative embodiment, catheter 14 includes a cable connector or interface 40, a handle 42, and a shaft 44 having a proximal end 46 and a distal 48 end. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

Localization and navigation system 30 may be provided for visualization, mapping and navigation of internal body structures. Localization and navigation system 30 may include conventional apparatus known generally in the art. For example, localization and navigation system 30 may be substantially similar to the EnSite Precision™ System, commercially available from Abbott Laboratories, and as generally shown in commonly assigned U.S. Pat. No. 7,263,397 titled “Method and Apparatus for Catheter Navigation and Location and Mapping in the Heart”, the entire disclosure of which is incorporated herein by reference. In another example, localization and navigation system 30 may be substantially similar to the EnSite X™ System, as generally shown in U.S. Pat. App. Pub. No. 2020/0138334 titled “Method for Medical Device Localization Based on Magnetic and Impedance Sensors”, the entire disclosure of which is incorporated herein by reference. It should be understood, however, that localization and navigation system 30 is an example only, and is not limiting in nature. Other technologies for locating/navigating a catheter in space (and for visualization) are known, including for example, the CARTO navigation and location system of Biosense Webster, Inc., the Rhythmia® system of Boston Scientific Scimed, Inc., the KODEX® system of Koninklijke Philips N.V., the AURORA® system of Northern Digital Inc., commonly available fluoroscopy systems, or a magnetic location system such as the gMPS system from Mediguide Ltd.

In this regard, some of the localization, navigation and/or visualization systems may include a sensor for producing signals indicative of catheter location information, and may include, for example, one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

Pulsed field ablation (PFA), which is a methodology for achieving irreversible electroporation, may be implemented using the systems and methods described herein. In some cases, PFA may be used at specific cardiac tissue sites such as the pulmonary veins to perform a pulmonary vein isolation (PVI). In PFA, electric fields may be applied between adjacent electrodes (in a bipolar approach) or between one or more electrodes and a return patch (in a monopolar approach). To monitor operation of system 10, one or more impedances between electrodes on catheter 14 and/or return electrodes 18, 20, and 21 may be measured. For example, for system 10, impedances may be measured as described in U.S. Patent Application Publication No. 2019/0117113, filed on October 23, 2018, U.S. Patent Application Publication No. 2019/0183378, filed on December 19, 2018, and U.S. Patent Application No. 63/027,660, filed on May 20, 2020, all of which are incorporated by reference herein in their entirety.

For electroporation therapy, waveforms are generated using a pulse generator (e.g., electroporation generator 26 (shown in FIG. 1)) and applied between pairs of catheter electrodes (i.e., a bipolar approach) or between catheter electrodes and a return patch (i.e., a monopolar approach). The waveforms may include one or more bursts of pulses (with each burst including multiple pulses). Further, the waveforms are defined by multiple parameters (e.g., pulse width, pulse amplitude, frequency, etc.

Different waveforms may be used to achieve different goals. For example, some waveforms may result in larger or smaller lesion size than other waveforms. Further, some waveforms result in higher or lower overall energy delivery than other waveforms (less overall energy delivery generally corresponds to less heating of the target tissue). As another example, some waveforms are more likely to induce muscular contractions in a patient. In general, however, electrical currents with a zero overall charge over the waveform may be used to prevent unwanted side-effects, as described herein. Generally, it is desirable to deliver electroporation therapy with a relatively low number of therapy applications over a relatively short timeframe. Further, it is generally desirable to avoid thermal heating of the tissue, and to have little to no skeletal muscle recruitment (i.e., avoiding muscle contractions.

The systems and method described herein use waveforms with a zero average charge and/or a reduced maximum absolute charge to reduce muscle contraction and/or nerve stimulation. The techniques described herein may be applied to waveforms with various frequencies, amplitudes, pulse shapes, initial pulse polarities, etc. Further, the embodiments described herein may be implemented using current sources, voltage sources, or intermediate output-impedance sources to generate the waveforms.

For the purposes of the discussion herein, an ideal amplifier is assumed, with no startup effects (i.e., the first cycle is identical to all following cycles), no close-down effects (i.e., the last cycle is identical to all previous cycles, no output after the last cycle), and infinite bandwidth. That being said, the waveforms described herein may be tuned to work with imperfect amplifiers, and circuits for implementing these waveforms can also be used to improve charge balancing.

In the following example waveforms, the horizontal axis represents time, and the vertical axis represents amplitude (e.g., in Ampere). Notably, the time scales, amplitudes, and pulse shapes shown are merely examples, and any suitable parameters may be used. Further, the amplitude may alternatively be expressed in current values, electrical field values, etc. For each example waveform, the waveform signal itself is shown, as well as an integrated value of the waveform signal (to illustrate the charge delivered by the waveform). The integrated value increases when the waveform signal is positive, is constant when the waveform signal is zero, and decreases when the waveform signal is negative.

The following example waveforms include positive and negative pulses. In some embodiments (e.g., for electroporation applications), a voltage amplitude of each pulse may be in a range from about 300 V to about 3,200 V, or more particularly, from about 1,000 V to about 2,000 V. Further, a pulse width of each individual pulse (i.e., a single positive pulse, or a single negative pulse) may be in a range from about 500 ns to 20 µs, more particularly, from about 1.0µs to about 10.0µs, and even more particularly, from about 2.0µs to about 5.0µs. Of course, those of skill in the art will appreciate that any suitable pulse parameters may be used.

For relatively low frequency signals, a neuron may fire on the first pulse of a burst. A neutralizing effect of a subsequent opposite polarity pulse (which returns the net charge to zero) may come too late to counter excitation. For such signals, the embodiments described herein address this problem by allowing twice the current without excitation by halving a maximum absolute value of the charge over a burst of pulses.

For high frequency signals, excitable tissue will experience roughly the average charge and will fire if roughly the average charge is above a certain level for a certain amount of time. Accordingly, many embodiments described herein have a zero average charge (as explained in detail below), thus reducing the chance of muscle and nerve stimulation for high frequency bursts.

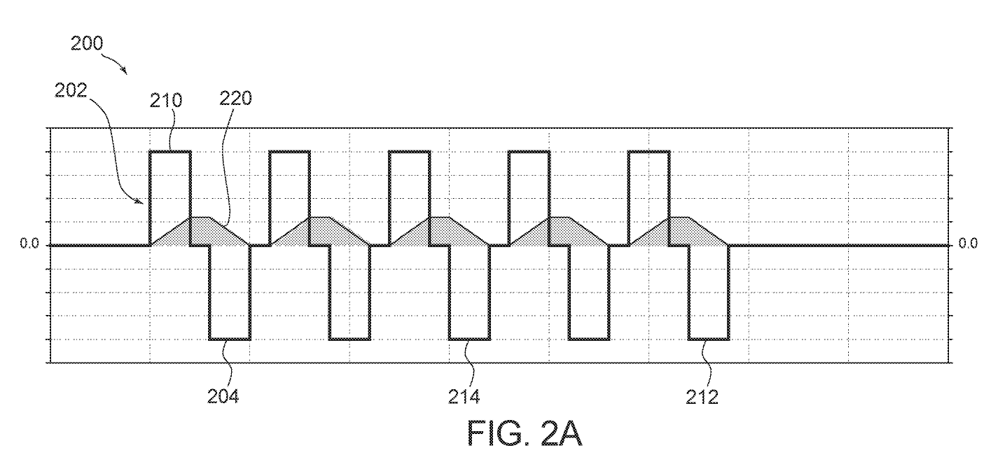

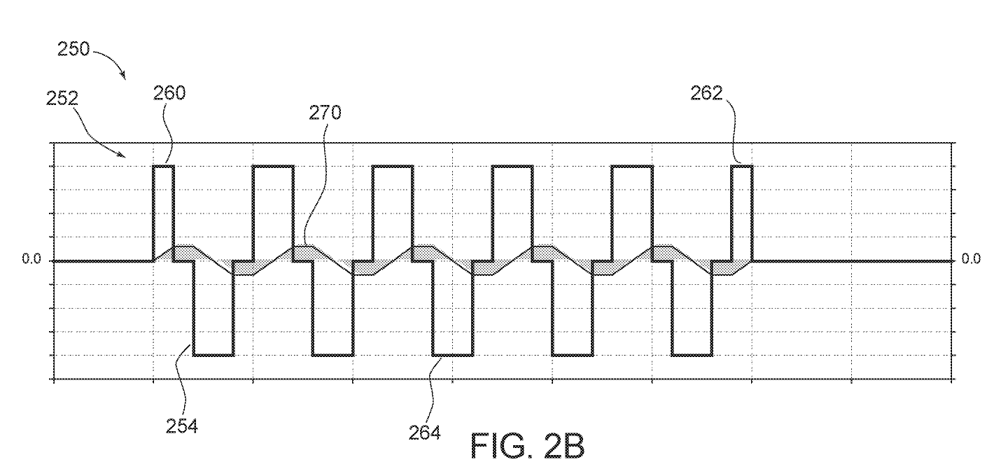

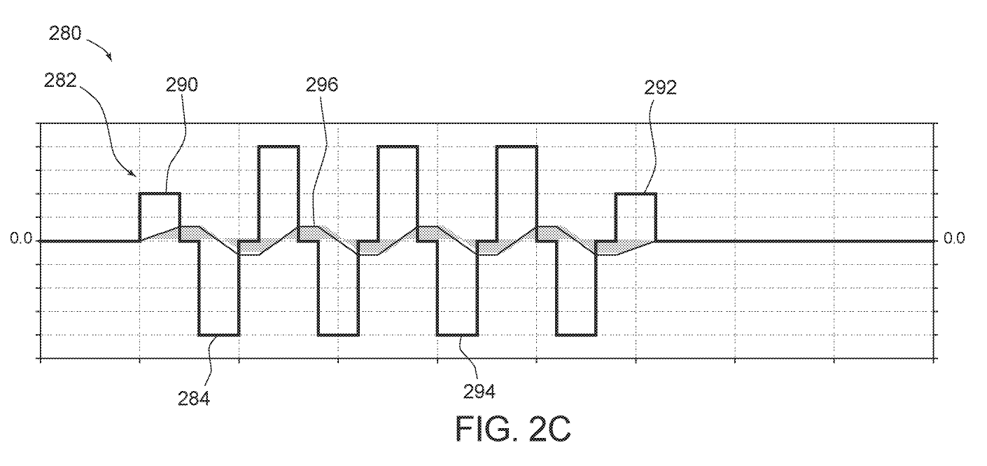

FIG. 2A is one embodiment of a waveform 200 that may be generated using system 10 (shown in FIG. 1). Waveform 200 includes a pulse train 202 having alternating positive and negative pulses 204. Pulse train 202 includes a first pulse 210, a last pulse 212, and a plurality of intermediate pulses 214. All pulses 204 have the same amplitude and pulse length. This may be referred to as a symmetric pulse pattern.

An integrated signal 220 represents the charge delivered by waveform 200 over time. As shown in FIG. 2A, the overall charge delivered by waveform 200 is zero (i.e., the charge at the end of the burst is zero). However, the average charge delivered by waveform 200 is non-zero at any time during the burst (i.e., the average charge is positive here, when calculated from the start of the burst until any time during the burst).

FIG. 2B is another embodiment of a waveform 250 that may be generated using system 10 (shown in FIG. 1). Waveform 250 includes a pulse train 252 having alternating positive and negative pulses 254. Pulse train 252 includes a first pulse 260, a last pulse 262, and a plurality of intermediate pulses 264.

In this embodiment, intermediate pulses 264 all have the same amplitude and pulse width. However, first pulse 260 and last pulse 262 have the same amplitude as intermediate pulses 264 (e.g., a voltage amplitude in a range from about 1000 V to about 2000 V), but have a different pulse width. Specifically, the pulse width of first pulse 260 and last pulse 262 is half the pulse width of intermediate pulses 264. For example, intermediate pulses 264 may have a pulse width of approximately 3.0µs, and first pulse 260 and last pulse 262 may have a pulse width of approximately 1.5µs. Further, first pulse 260 and last pulse 262 have the same polarity.

An integrated signal 270 represents the charge delivered by waveform 250 over time. By halving the pulse width of first pulse 260 and last pulse 262 (relative to waveform 200), the average charge delivered by waveform 250 is zero. That is, the average value of integrated signal 270 is zero over the burst as a whole, and also at regular intervals throughout the burst. Further, the overall charge delivered by waveform 250 is zero, demonstrated by integrated signal 270 being zero at the end of waveform 250.

Notably, the maximum magnitude of integrated signal 270 (corresponding to the maximum absolute charge value) is less than the maximum magnitude of integrated signal 220. This reduced magnitude reduces the chance for muscle contraction and/or nerve stimulation. This reduced magnitude may also facilitate using electrodes with smaller surfaces in some applications.

FIG. 2C is another embodiment of a waveform 280 that may be generated using system 10 (shown in FIG. 1). Waveform 280 includes a pulse train 282 having alternating positive and negative pulses 284. Pulse train 282 includes a first pulse 290, a last pulse 292, and a plurality of intermediate pulses 294.

In this embodiment, intermediate pulses 294 all have the same amplitude and pulse width. However, first pulse 290 and last pulse 292 have the same pulse width as intermediate pulses 294 (e.g., a pulse width of approximately 3.0µs) but have a different amplitude. Specifically, the pulse amplitude of first pulse 290 and last pulse 292 is half the amplitude of intermediate pulses 294. For example, intermediate pulses 294 may have a voltage amplitude of approximately 1500 V, and first pulse 290 and last pulse 292 may have a voltage amplitude of approximately 750 V. Further, first pulse 290 and last pulse 292 have the same polarities.

An integrated signal 296 represents the charge delivered by waveform 280 over time. By halving the amplitudes of first pulse 290 and last pulse 292 (relative to waveform 200), the average charge delivered by waveform 280 is zero. That is, the average value of integrated signal 296 is zero over the burst as a whole, as well as at regular intervals throughout the burst. Further, the overall charge delivered by waveform 280 is zero, demonstrated by integrated signal 296 being zero at the end of waveform 280.

Again, the maximum magnitude of integrated signal 296 (corresponding to the maximum absolute charge value) is less than the maximum magnitude of integrated signal 220. This reduced magnitude reduces the chance for muscle contraction and/or nerve stimulation. This reduced magnitude may also facilitate using electrodes with smaller surfaces in some applications.

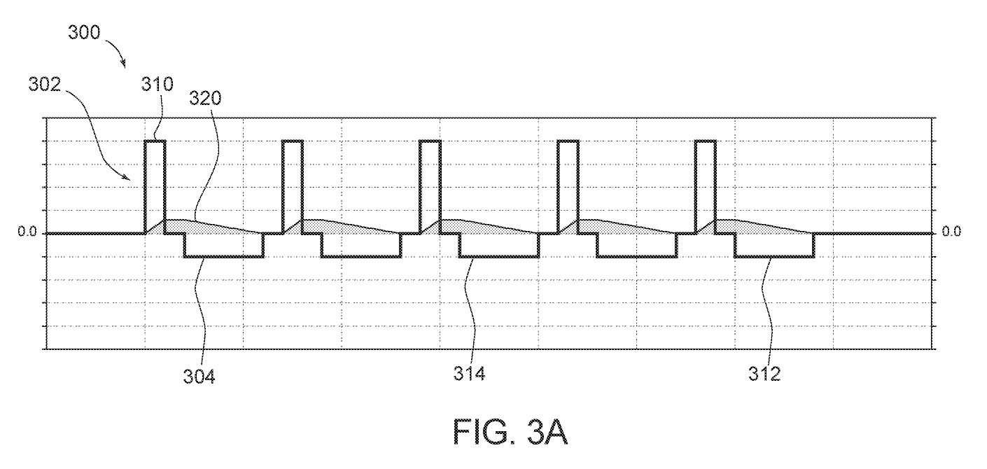

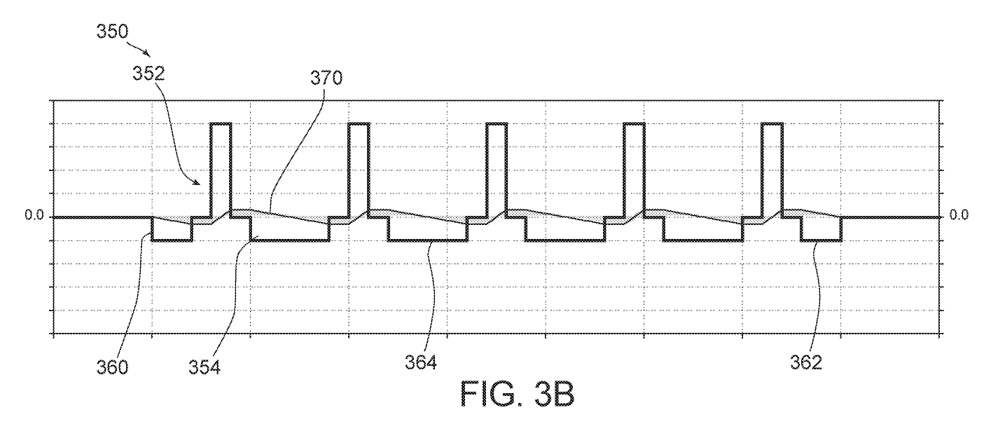

FIG. 3A is another embodiment of a waveform 300 that may be generated using system 10 (shown in FIG. 1). Waveform 300 includes a pulse train 302 having alternating positive and negative pulses 304. Pulse train 302 includes a first pulse 310, a last pulse 312, and a plurality of intermediate pulses 314. In one example, first pulse 310, last pulse 312, and intermediate pulses 314 each have a voltage amplitude in a range from about 200 V to about 2000 V, and a pulse width in a range from about 1.0µs to about 5.0µs. Alternatively, any suitable voltage and pulse width values may be used. All positive pulses in pulse train 302 have a larger amplitude and shorter pulse length than all negative pulses in pulse train 302. However, the area under each positive pulse is the same as the area under each negative pulse. This may be referred to as an asymmetric pulse pattern.

An integrated signal 320 represents the charge delivered by waveform 300 over time. As shown in FIG. 3A, the overall charge delivered by waveform 300 is zero, due to the areas under the positive and negative pulses being equal. However, the average charge delivered by waveform 300 is non-zero at any time during the burst. Accordingly, waveform 300 will result in an “average charge” being experienced by the tissue throughout the burst.

FIG. 3B is another embodiment of a waveform 350 that may be generated using system 10 (shown in FIG. 1). Waveform 350 includes a pulse train 352 having alternating positive and negative pulses 354. Pulse train 352 includes a first pulse 360, a last pulse 362, and a plurality of intermediate pulses 364. In one example, first pulse 360, last pulse 362, and intermediate pulses 364 each have a voltage amplitude in a range from about 200 V to about 2000 V, and a pulse width in a range from about 1.0µs to about 5.0µs. Alternatively, any suitable voltage and pulse width values may be used.

In this embodiment, positive pulses of intermediate pulses 364 have a larger amplitude and shorter pulse length than negative pulses of intermediate pulses 364. Further, first pulse 360 and last pulse 362 have the same polarity. Notably, first pulse 360 and last pulse 362 have the same amplitude as intermediate pulses 364 of the same polarity (i.e., negative intermediate pulses 364) but have a different pulse width. Specifically, the pulse width of first pulse 360 and last pulse 362 is half the pulse width of negative intermediate pulses 364.

An integrated signal 370 represents the charge delivered by waveform 350 over time. By halving the pulse width of first pulse 360 and last pulse 362 (relative to waveform 300), the average charge delivered by waveform 350 is zero. That is, the average value of integrated signal 370 is zero over the burst as a whole, as well as at regular intervals throughout the burst. Further, the overall charge delivered by waveform 350 is zero, demonstrated by integrated signal 370 being zero at the end of waveform 350.

Notably, the maximum magnitude of integrated signal 370 (corresponding to the maximum absolute charge value) is less than the maximum magnitude of integrated signal 320. This reduced magnitude reduces the chance for muscle contraction and/or nerve stimulation. This reduced magnitude may also facilitate using electrodes with smaller surfaces in some applications.

By using the polarity that has the longer pulse width (i.e., the negative pulses in this example) for first pulse 360 and last pulse 362, demands on bandwidth and switching speed of the amplifier are reduced.

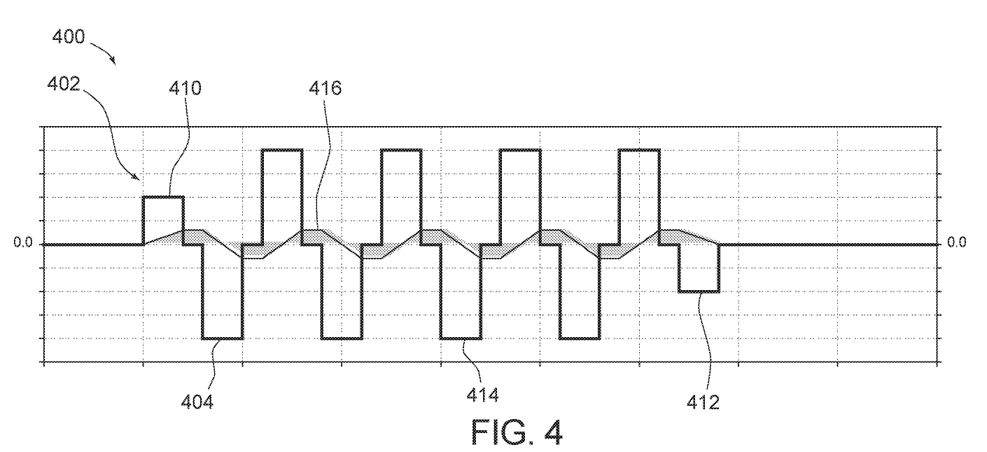

FIG. 4 is another embodiment of a waveform 400 that may be generated using system 10 (shown in FIG. 1). Waveform 400 includes a pulse train 402 having alternating positive and negative pulses 404. Pulse train 402 includes a first pulse 410, a last pulse 412, and a plurality of intermediate pulses 414. In one example, first pulse 410, last pulse 412, and intermediate pulses 414 each have a voltage amplitude in a range from about 200 V to about 2000 V, and a pulse width in a range from about 1.0µs to about 5µs. Alternatively, any suitable voltage and pulse width values may be used.

In this embodiment, intermediate pulses 414 all have the same amplitude and pulse width. However, first pulse 410 and last pulse 412 have the same pulse width as intermediate pulses 414 but have a different amplitude. Specifically, the pulse amplitude of first pulse 410 and last pulse 412 is half the amplitude of intermediate pulses 414. Accordingly, waveform 400 is somewhat similar to waveform 280 (shown in FIG. 3C). However, unlike waveform 280, first pulse 410 and last pulse 412 have different polarities.

An integrated signal 416 represents the charge delivered by waveform 400 over time. For waveform 400, the average charge delivered is non-zero over the burst as a whole, but still zero at regular intervals throughout the burst. Further, the overall charge delivered by waveform 400 is still zero, demonstrated by integrated signal 416 being zero at the end of waveform 400.

For waveform 400, although the average delivered is non-zero, the maximum magnitude of integrated signal 416 (corresponding to the maximum absolute charge value) is still less than the maximum magnitude of the integrated signal 200. The reduced magnitude reduces the chance for muscle contraction and/or nerve stimulation. This reduced magnitude may also facilitate using electrodes with smaller surfaces in some applications.

Instead of first pulse 410 and last pulse 412 having half the amplitude of intermediate pulses 414, as will be appreciated by those of skill in the art, first pulse 410 and last pulse 412 may have the same amplitude as intermediate pulses 414, but half the pulse width of intermediate pulses 414 to achieve similar results.

FIGS. 2B, 2C, and 3B show waveforms that constitute a complete burst of pulses (e.g., including multiple positive and negative pulses). However, those of skill in the art will appreciate that the techniques described herein (e.g., achieving zero average charge over a waveform) may be implemented with fewer pulses. For example, a subset of pulses within a burst may constitute a waveform with zero average charge, while the burst itself does not have zero average charge.

In the embodiments disclosed herein, short periods between positive and negative pulses allow for timing inaccuracies in switching components and decrease bandwidth of the output signal. These periods do not affect the overall average charge (i.e., zero), nor the reduction of the maximum absolute charge. In some embodiments, pulse length is tuned to account for non-ideal switching times or non-resistive loads. Further, circuits used to implement the first and last pulses can be used to correct for amplifier imperfections (e.g., turn-on effects at the start of burst or amplitude decreases towards the end of the burst) to improve charge balancing.

Those of skill in the art will appreciate that the specific waveforms disclosed herein are examples. In general, any combination of pulse duration, pulse amplitude, or pulse shape can be used to cause the first and last pulses to achieve half the charge level of the intermediate pulses, resulting in a zero average charge over the waveform. That is, the waveform having zero average charge may be included within and have a shorter duration than a longer burst waveform.

The systems and methods described herein are directed to a pulse generator for use with an electroporation system. The pulse generator is configured to be coupled to a catheter including a plurality of electrodes and is configured to generate a waveform to be delivered using at least one of the plurality of electrodes. The waveform includes a pulse train having positive and negative pulses with at least one of a reduced maximum absolute charge and a zero average charge over the pulse train.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader’s understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

1. A pulse generator for use with an ablation system, the pulse generator configured to be coupled to a catheter including at least one electrode and configured to generate a waveform to be delivered using the at least one electrode, the waveform including:
a pulse train including a first pulse having a first polarity, at least one intermediate pulse having a second, opposite polarity, and a last pulse having the first polarity, wherein the overall charge delivered by the pulse train is zero.

2. The pulse generator in accordance withclaim 1, wherein the first polarity is negative and the second polarity is positive.

3. The pulse generator in accordance with claim 1, wherein the first pulse and the second pulse have a first pulse width, wherein the at least one intermediate pulse has a second pulse width, and wherein the first pulse width is greater than the second pulse width.

4. The pulse generator in accordance with claim 1, wherein the first pulse and the second pulse have a first pulse amplitude, wherein the at least one intermediate pulse has a second pulse amplitude, and wherein the first pulse amplitude is less than the second pulse amplitude.

5. The pulse generator in accordance with claim 1, wherein the at least one intermediate pulse comprises a single intermediate pulse.

6. The pulse generator in accordance with claim 1, wherein the waveform is a monopolar waveform.

7. The pulse generator in accordance with claim 1, wherein an average charge over the pulse train is zero.

8. The pulse generator in accordance with claim 1, wherein each of the pulses in the pulse train have a pulse width in a range from about 1.0 µs to about 5.0 µs.

9. The pulse generator in accordance with claim 1, wherein the first pulse and the last pulse have a pulse amplitude in a range from about 200 V to about 2000 V.

10. An ablation system comprising:
a pulse generator configured to generate a waveform; and
a catheter coupled to the pulse generator and including at least one electrode configured to deliver the generated waveform, wherein the waveform includes:
a pulse train including a first pulse having a first polarity, at least one intermediate pulse having a second, opposite polarity, and a last pulse having the first polarity, wherein the overall charge delivered by the pulse train is zero.

11. The ablation system in accordance with claim 10, wherein the first polarity is negative and the second polarity is positive.

12. The ablation system in accordance with claim 10, wherein the first pulse and the second pulse have a first pulse width, wherein the at least one intermediate pulse has a second pulse width, and wherein the first pulse width is greater than the second pulse width.

13. The ablation system in accordance with claim 10, wherein the first pulse and the second pulse have a first pulse amplitude, wherein the at least one intermediate pulse has a second pulse amplitude, and wherein the first pulse amplitude is less than the second pulse amplitude.

14. The ablation system in accordance with claim 10, wherein the at least one intermediate pulse comprises a single intermediate pulse.

15. The ablation system in accordance with claim 10, wherein the waveform is a monopolar waveform.

16. The ablation system in accordance with claim 10, wherein an average charge over the pulse train is zero.

17. The ablation system in accordance with claim 10, each of the pulses in the pulse train have a pulse width in a range from about 1.0 µs to about 5.0 µs.

18. A method for controlling an ablation system, the method comprising:
generating, using a pulse generator, a waveform including a pulse train including a first pulse having a first polarity, at least one intermediate pulse having a second, opposite polarity, and a last pulse having the first polarity, wherein the overall charge delivered by the pulse train is zero;
delivering, using at least one electrode on a catheter coupled to the pulse generator, the generated waveform to target tissue.

19. The method in accordance with claim 18, wherein the first polarity is negative and the second polarity is positive.

20. The method in accordance with claim 18, wherein the first pulse and the second pulse have a first pulse width, wherein the at least one intermediate pulse has a second pulse width, and wherein the first pulse width is greater than the second pulse width.
